(19) United States
(12) Patent Application Publication
(10) Pub. No.: US 2005/0065558 A1
(43) Pub. Date: Mar. 24, 2005

(54) External defibrillator having a removable battery pack using off-the-shelf cells

(76) Inventors: Daniel J. Powers, Issaquah, WA (US); Anthony George Picardo, Tacoma, WA (US); Jon Mikalson Bishay, Woodinville, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/895,230

(22) Filed: Jul. 20, 2004

Related U.S. Application Data
(60) Provisional application No. 60/504,406, filed on Sep. 19, 2003.

(51) Int. Cl. A61N 1/39

(52) U.S. Cl. 607/5

(57) ABSTRACT

In accordance with the present invention, an electrotherapy device (12) includes a controller (44), an energy source (42), at least one electrode (46) for providing electrotherapy to a patient (48), an energy delivery system (49) operable by the controller to deliver an electrical shock from the energy source (42) to the electrode (46), and a removable battery pack (10) that serves as the energy source (42). The removable battery pack (10) includes a housing having a user accessible interior and an exterior configured to be removably insertable into a battery receptacle (20) of the electrotherapy device (12). The battery pack (10) also includes at least one user replaceable, non-rechargable cell (18) located in the interior of the housing. The cell (18) has a positive and negative terminal. A pair of conductive contacts is located in the interior of the housing. One of the conductive contacts is coupled to the positive terminal of the cell (18) and the other conductive contact is coupled to the negative terminal of the cell (18). A battery pack circuit (8) located in the interior of the housing is in electrically operative communication with the cell (18).

This invention claims the benefit of Provisional U.S. Patent Application Ser. No. 60/504,406, filed Sep. 19, 2003.

The present invention relates generally to external defibrillators, and more particularly to a power source for an external defibrillator.

Sudden cardiac death is the leading cause of death in the United States, with one person dying every two minutes. Most sudden cardiac death is caused by ventricular fibrillation (“VF”), in which the heart's muscle fibers contract without coordination, thereby interrupting normal blood flow to the body. The only known effective treatment for VF is electrical defibrillation, in which an electrical pulse is applied to the patient's heart. The electrical pulse must be delivered within a short time after onset of VF in order for the patient to have any reasonable chance of survival. Electrical defibrillation may also be used to treat shockable ventricular tachycardia (“VT”). Accordingly, defibrillation is the appropriate therapy for any shockable rhythm, i.e., VF or shockable VT.

One way of providing electrical defibrillation uses an external defibrillator. External defibrillators send electrical pulses to the patient's heart through electrodes applied to the patient's torso. External defibrillators are typically located and used in hospital emergency rooms, operating rooms, and emergency medical vehicles. Of the wide variety of external defibrillators currently available, automatic and semi-automatic external defibrillators (referred to collectively as “AEDs”) are becoming increasingly popular because they can be used by relatively inexperienced personnel. Such AEDs are also especially lightweight, compact, and portable. AEDs are described in U.S. Pat. No. 5,607,454 to Cameron et al. entitled “Electrotherapy Method and Apparatus,” PCT Publication No. WO 94/27674 entitled “Defibrillator with Self-Test Features,” and U.S. Published Patent Application No. 2002/0156503 entitled “Method and Apparatus for Providing On-Screen Incident Review in an AED,” the specifications of which are incorporated herein.

All defibrillators must contain or be connected to an energy source to generate and apply a defibrillation pulse to the patient. To ensure true portability, most portable defibrillators are constructed with a battery pack that is sufficient to operate the portable defibrillator for a period of time. The battery pack may be either rechargeable or non-rechargeable, depending on the user's preference and the environment in which the defibrillator is to be used. Rechargeable battery packs typically power a defibrillator for a shorter period of time than non-rechargeable battery packs, but can be recharged and reused. In contrast, non-rechargeable battery packs allow the defibrillator to operate for a longer period but require replacement when the battery pack is discharged.

While battery packs are often more expensive than conventional batteries, the use of battery packs may be preferable for a number of reasons. For example, a battery pack may include storage cells designed to satisfy the particular power requirements of a device with which the battery pack will be used. Battery packs can also be designed to be coupled to the device more quickly, easily, and effectively than conventional batteries. Further, along with the storage cells, a battery pack may house additional components useful to the device and unavailable with standard batteries. For example, the battery pack may include circuitry associated with the cells. In particular, the cells may be connected to a safety circuit contained on a circuit board within the battery pack. Finally, the battery pack housing may provide better mechanical and environmental protection for the storage cells than is available with conventional batteries.

A typical conventional battery pack includes a housing that is formed of injection-molded plastic, for example, and which is bonded together by ultrasonic welding. The heart of the battery pack is two or more cells (and typically four or more) that are connected in series or parallel with one another. The voltage of each cell is selected to satisfy the power requirements of the defibrillator. The type of cell is selected depending upon the particular requirements of the user, and environment in which the defibrillator will be used. For example, the cells may be non-rechargeable cells such as lithium-sulfur dioxide (LiSO₂) cells that each generate three volts, with the series connection of the four lithium cells generating 12 volts. Lithium cells are non-rechargeable, but have a very high energy density. A lithium battery pack will therefore ensure an extended operating time for the defibrillator. Alternatively, the cells may be Lithium Manganese Dioxide (LiMNO₂) cells.

One problem with a conventional non-rechargable battery pack is that when the lithium cells are nearly discharged, the entire battery pack must be discarded. One conventional defibrillator overcomes this problem by using commercially available, user-replaceable cells. The cells are installed directly into the defibrillator rather than in a pack or tray. A problem with this approach is that the cells may need to be replaced during an emergency in which the defibrillator is to be used. Such an emergency is very stressful and responders are often not thinking clearly when it occurs. Accordingly, it is particularly important that the cells be capable of easy removal and replacement during an emergency without any loss of precious time. This can be problematic if the cells are installed directly into the defibrillator unit since there are typically many cells that need to be replaced, at high speed and with exact precision. For example, if even one cell were inadvertently installed backward, a victim could potentially die before the problem was identified and corrected.

In accordance with the present invention, an electrotherapy device includes a controller, an energy source, at least one electrode for providing electrotherapy to a patient, an energy delivery system operable by the controller to deliver an electrical shock from the energy source to the electrode, and a removable battery pack that serves as the energy source. The removable battery pack includes a housing having a user accessible interior and an exterior configured to be removably insertable into a battery receptacle of the electrotherapy device. The battery pack also includes at least one user replaceable, non-rechargable cell located in the interior of the housing. The cell has a positive and negative terminal. A pair of conductive contacts is located in the interior of the housing. One of the conductive contacts is coupled to the positive terminal of the cell and the other conductive contact is coupled to the negative terminal of the cell. A battery pack circuit located in the interior of the housing is in electrically operative communication with the cell.

In accordance with one aspect of the invention, the housing has a top, a base, and an arrangement for interlocking the top to the base.

In accordance with another aspect of the invention, the interlocking arrangement includes a plurality of latches.

In accordance with another aspect of the invention, the housing includes a latch for inserting and removing the battery pack from the battery pack receptacle.

In accordance with another aspect of the invention, the battery pack circuit comprises a safety circuit.

In accordance with another aspect of the invention, an arrangement is provided for verifying proper reception of the user replaceable, non-rechargeable cell.

In accordance with another aspect of the invention, the verifying arrangement comprises illustrative indications disposed on the housing to indicate proper installation.

In accordance with another aspect of the invention, the verifying arrangement comprises a keying surface disposed on the interior which prevents the cell from being received into the battery pack incorrectly.

In accordance with another aspect of the invention, the verifying arrangement comprises a voltage verification circuit in electrically operative communication with the cell.

In accordance with another aspect of the invention, the voltage verification circuit is disposed on the housing.

In accordance with another aspect of the invention, the voltage verification circuit is disposed on the external defibrillator.

In accordance with another aspect of the invention, a removable battery pack is provided for an external defibrillator. The battery pack includes a housing having a user accessible interior and an exterior configured to be removably insertable into a battery receptacle of the external defibrillator. The interior is disposed to receive at least one user replaceable, non-rechargeable cell.

In accordance with another aspect of the invention, the housing has a top, a base, and an arrangement for interlocking the top to the base.

In accordance with another aspect of the invention, the interlocking arrangement comprises a plurality of latches.

In accordance with another aspect of the invention, the housing further comprises a latch for inserting and removing the battery pack from the battery pack receptacle.

In accordance with another aspect of the invention, a battery pack circuit is located in the interior of the housing and is in electrically operative communication with the cell.

In accordance with another aspect of the invention, the battery pack circuit comprises a safety circuit.

In accordance with another aspect of the invention, a removable battery pack is provided for an external defibrillator. The battery pack includes a housing having a user accessible interior and an exterior configured to be removably insertable into a battery receptacle of the external defibrillator. At least one user replaceable, non-rechargable cell is located in the interior of the housing. The cell has a positive and negative terminal. A pair of conductive contacts is located in the interior of the housing. One of the pair of conductive contacts is coupled to the positive terminal of the cell and the other of the pair of conductive contacts is coupled to a negative terminal of the cell. A battery pack circuit is located in the interior of the housing and is in electrically operative communication with the cell.

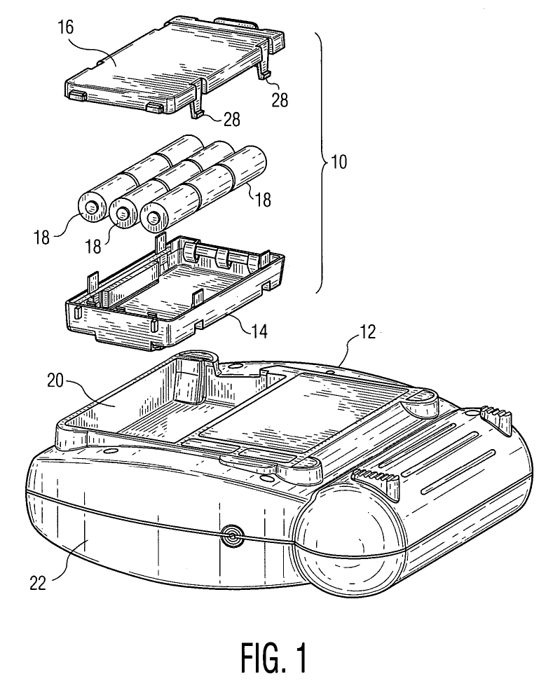

FIG. 1 shows a defibrillator and an exploded view of a battery pack for use in the defibrillator in accordance with the present invention.

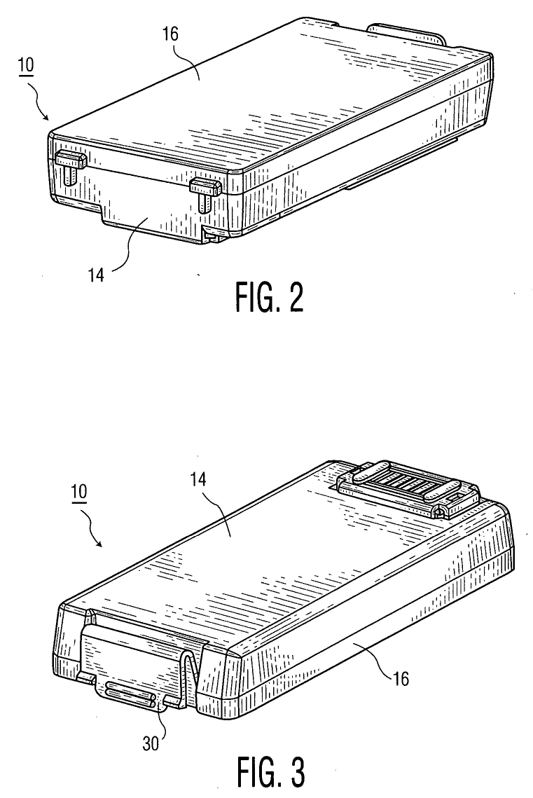

FIGS. 2 and 3 show various perspective views of the battery pack shown in FIG. 1.

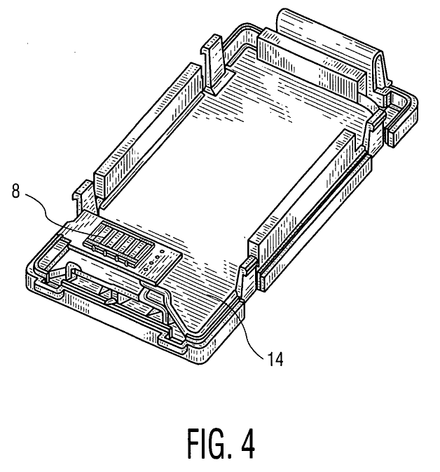

FIG. 4 shows the interior of the battery pack base.

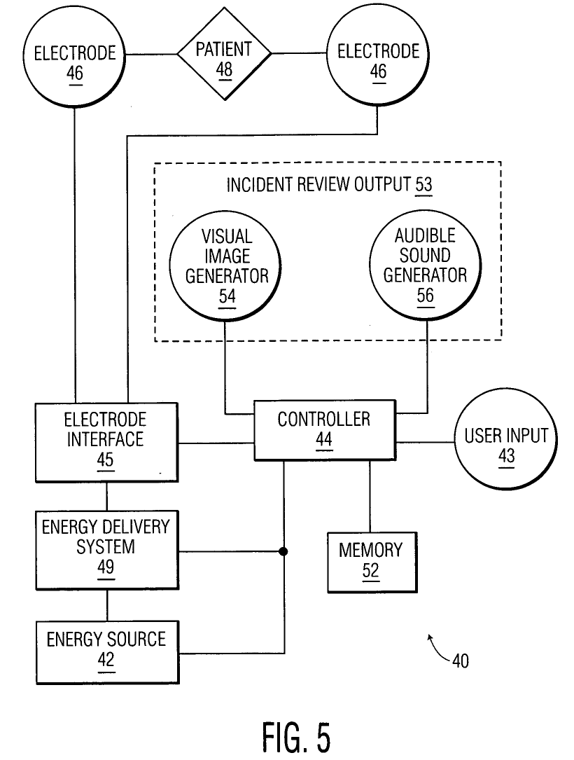

FIG. 5 is a schematic block diagram of a defibrillator in which the present invention may be employed.

The following discussion is presented to enable a person skilled in the art to make and use the invention. Various modifications to the preferred embodiment will be readily apparent to those skilled in the art, and the generic principles defined herein may be applied to other embodiments and applications without departing from the spirit and scope of the present invention as defined by the appended claims. Thus, the present invention is not intended to be limited to the embodiment shown, but is to be accorded the widest scope consistent with the principles and features disclosed herein.

A defibrillator battery pack of the present invention is preferably adapted for use with an automated external defibrillator (AED). An AED 12 in accordance with the present invention is illustrated generally in FIG. 1. Defibrillator 12 includes plastic case 22, and a pair of electrodes (not shown) for placement on a patient for delivering a defibrillation shock. Defibrillator battery pack 10 of the present is removably insertable into battery receptacle 20 of AED plastic case 22.

The present inventors have recognized the desirability of providing a non-rechargable battery pack for a defibrillator that employs readily available, user-replaceable cells. Since the cells are installed in a battery pack and not directly into the defibrillator unit, it is possible to maintain a spare pack in which the cells are installed in advance of an emergency. In this way a depleted battery pack can be quickly and easily exchanged with the spare pack, while still obtaining the benefits such as ease and convenience that arise from the provision of user-replaceable cells.

FIG. 1 shows an exploded view of one embodiment of a battery pack 10 constructed in accordance with the present invention. The battery pack 10 is a portable power source that provides the energy required by defibrillator 12. The battery pack 10 comprises a housing having a base 14 and cover 16, which may be formed of injection-molded plastic or other materials. The base 14 and cover 16 cooperatively protect the other components of the battery pack 10 both mechanically and electrically. In addition, the base 14 plays a key role in the alignment, latching and ejection of the battery pack 10 relative to the defibrillator 12. Four snap-type latches 28 are provided on the cover 16, which interlock with the base 14 so that the battery pack 10 can be easily assembled and disassembled by the user. As best seen in FIG. 3, another latch 30, which is located on the exterior of the base 14, is provided for inserting and removing the battery pack 10 from its receptacle 20 in the defibrillator 12.

The battery pack 10 may comprise an illustrative figure to indicate the proper positioning and orientation of the cell or cells in the pack. The illustrative figure may be drawn on the base 14 or cover 16.

The inside surface of base 14 or cover 16 may also include a keying surface such that the battery pack 10 cannot be assembled if the cell or cells are improperly installed. For example, the keying surface may comprise an indent to receive the anode protrusion of a cell 18. Installation of cell 18 with its cathode side to the key would not be possible due to the longer installed length of cell 18.

The battery pack 10 includes cells 18 that are connected in series and/or in parallel with one another. The number of cells 18 and the voltage of each cell is selected to satisfy the power requirements of defibrillator 12. The type of cell is selected depending upon the particular requirements of the user and the environment in which the defibrillator will be used. Regardless of the type and voltage and number of cells that are employed, cells 18 are commercially available cells that can be replaced by the user. For example, in the embodiment of the invention shown in FIG. 1, nine cells 18 in a A size are employed. Three sets or strings of three cells each are connected in series. The three strings are then connected to one another in parallel. The cells 18 are Lithium Manganese Dioxide (LiMNO₂) cells that each three generate volts, with the series connection of the three cells generating 9 volts per string.

As seen in FIG. 4, a battery pack circuit 8 is provided on a circuit board that is located in the base 14. In the embodiment of the invention shown in the figures, the circuit 8 connects the three sets or strings of cells to the battery contact arrangement. The battery contact arrangement has four contacts. One of the contacts is a common negative terminal for all three strings. Each string of cells is then respectively connected to the remaining three contacts.

The battery pack circuit 8 may provide a variety of safety features for the defibrillator. For example, wires from the cells may be connected to two fuses contained on the circuit board. The first fuse may be a current fuse that will trip if sufficient current flows through the circuit. Undue current flow from the cells may be caused by a short within the defibrillator or a short across the conductive pins on the exterior of the battery pack. The second fuse may be a thermal fuse that will trip when the temperature within the battery pack reaches a predetermined threshold. The thermal fuse will therefore trip due to certain types of cell failure within the battery pack, or other environmental condition causing the temperature within the battery pack to unduly rise. Wires from the cells and circuitry connect to connectors and connective pins that are located on the external surface of the battery pack housing. Current therefore flows from the cells to the defibrillator through the safety circuit and the connectors.

The battery pack circuit may also comprise a voltage verification circuit in electrical communication with the cells. The voltage verification circuit monitors cell voltages to provide immediate feedback that the user has installed the cells properly. For example, the verification circuit could measure the voltage between connectors and ground. If the measured voltage is not an expected voltage, the circuit causes an appropriate audible or visible alert to be issued. Preferably, the verification circuit is enabled only during and shortly after cell insertion, such that the installer has immediate feedback on proper installation. The existing defibrillator battery self check feature is subsequently responsible for monitoring battery status over long periods of time. Alternatively, the verification circuit can be disposed in the defibrillator circuitry, and provide the same verification using existing defibrillator audible and visual alerts.

FIG. 5 is a schematic block diagram of a defibrillator (e.g., AED 12 in FIG. 1) in which the present invention may be employed. The defibrillator system 40 comprises an energy source 42 such as the inventive battery pack to provide voltage or current pulses. A controller 44, coupled to user input 43, operates an energy delivery system 49 to selectively connect and disconnect energy source 42 to and from a pair of electrodes 46 electrically attached to a patient 48 through an electrode interface 45 to provide electrotherapy to the patient. The defibrillator system 40 is an electrotherapy device such as a manual defibrillator or AED. A memory 52 records data collected by the defibrillator while monitoring and treating a patient. The data may also be reviewed through an incident review output 53, which may consist of a display 54 and an audible sound generator 56.

Although various embodiments are specifically illustrated and described herein, it will be appreciated that modifications and variations of the present invention are covered by the above teachings and are within the purview of the appended claims without departing from the spirit and intended scope of the invention. For example, the defibrillator battery pack need not employ the particular latching mechanisms depicted in the figures, but more generally may encompass any appropriate means for interlocking the components of the battery pack to one another and to the defibrillator itself. Moreover, the shape and dimensions of the battery pack may differ from those shown herein.

1. In an electrotherapy device (12) that includes a controller (44), an energy source (42) at least one electrode (46) for providing electrotherapy to a patient (48), an energy delivery system (49) operable by the controller (44) to deliver an electrical shock from the energy source (42) to the at least one electrode (46), a removable battery pack (10) that serves as the energy source (42), comprising;
a housing having a user accessible interior and an exterior configured to be removably insertable into a battery receptacle (20) of the electrotherapy device (12);
at least one user replaceable, non-rechargable cell (18) located in the interior of the housing, said cell (18) having a positive and negative terminal;
a pair of conductive contacts located in the interior of the housing, one of the pair of conductive contacts being coupled to the positive terminal of the cell (18) and the other of the pair of conductive contacts being coupled to a negative terminal of the cell (18); and
a battery pack circuit (8) located in the interior of the housing and in electrically operative communication with the cell (18).

2. In the electrotherapy device (12) of claim 1, a removable battery pack (10) wherein said housing has a top (16), a base (14), and means for interlocking the top (16) to the base (14).

3. In the electrotherapy device (12) of claim 2, a removable battery pack (10) wherein said interlocking means comprises a plurality of latches (28).

4. In the electrotherapy device (12) of claim 1, a removable battery pack (10) wherein said housing further comprises a latch (30) for inserting and removing the battery pack (10) from the battery pack receptacle (20).

5. In the electrotherapy device (12) of claim 1, a removable battery pack (10) wherein said battery pack circuit (8) comprises a safety circuit.

6. In the electrotherapy device (12) of claim 1, a removable battery pack (10) of claim 6 further comprising means for verifying proper reception of said user replaceable, non-rechargeable cell (18).

7. A removable battery pack (10) for an external defibrillator (12), comprising;
a housing having a user accessible interior and an exterior configured to be removably insertable into a battery receptacle (20) of the external defibrillator (12); wherein said interior is disposed to receive at least one user replaceable, non-rechargeable cell (18).

8. The removable battery pack (10) of claim 7 wherein said housing has a top (16), a base (14), and means for interlocking the top (16) to the base (14).

9. The removable battery pack (10) of claim 8 wherein said interlocking means comprises a plurality of latches (28).

10. The removable battery pack (10) of claim 7 wherein said housing further comprises a latch (30) for inserting and removing the battery pack (10) from the battery pack receptacle (20).

11. The removable battery pack (10) of claim 7 further comprising a battery pack circuit (8) located in the interior of the housing and in electrically operative communication with the cell (18).

12. The removable battery pack (10) of claim 11 wherein said battery pack circuit (8) comprises a safety circuit.

13. The removable battery pack (10) of claim 7 further comprising means for verifying the proper reception of said user replaceable, non-rechargeable cell (18).

14. A removable battery pack (10) for an external defibrillator (12), comprising;
a housing having a user accessible interior and an exterior configured to be removably insertable into a battery receptacle (20) of the external defibrillator (12);
at least one user replaceable, non-rechargable cell (18) located in the interior of the housing, said cell (18) having a positive and negative terminal;
a pair of conductive contacts located in the interior of the housing, one of the pair of conductive contacts being coupled to the positive terminal of the cell (18) and the other of the pair of conductive contacts being coupled to a negative terminal of the cell (18); and
a battery pack circuit (8) located in the interior of the housing and in electrically operative communication with the cell (18).

15. The removable battery pack (10) of claim 14 wherein said housing has a top (16), a base (14), and means for interlocking the top (16) to the base (14).

16. The removable battery pack (10) of claim 15 wherein said interlocking means comprises a plurality of latches (28).

17. The removable battery pack (10) of claim 14 wherein said housing further comprises a latch (30) for inserting and removing the battery pack (10) from the battery pack receptacle (20).

18. The removable battery pack (10) of claim 14 wherein said battery pack circuit (8) comprises a safety circuit.
